(19) United States
(12) Patent Application Publication
(10) Pub. No.: US 2018/0311137 A1
(43) Pub. Date: Nov. 1, 2018

(54) LOTION COMPOSITIONS COMPRISING SOIL CAPTURE POLYMERS

(71) Applicant: The Procter & Gamble Company, Cincinnati, OH (US)

(72) Inventors: Robin Lynn McKIERNAN, Mason, OH (US); Randall Glenn MARSH, Hamilton, OH (US); Steven Daryl SMITH, Fairfield, OH (US); Jamie Lynn DRIA, Deerfield Township, OH (US); Robert Joseph McCHAIN, Cincinnati, OH (US)

(21) Appl. No.: 15/964,097

(22) Filed: Apr. 27, 2018

Related U.S. Application Data
(60) Provisional application No. 62/491,286, filed on Apr. 28, 2017.

(51) Int. Cl. A61K 8/81; A61Q 19/00; A61Q 19/10; A61K 8/02

(52) U.S. Cl. CPC A61K 8/8158; A61Q 19/007; A61K 2800/5426; A61Q 19/008; A61K 8/0208; A61Q 19/10

(57) ABSTRACT

Described herein is a lotion composition including a soil capture polymer, the soil capture polymer having a number average molecular weight of at least 1,000,000 and a cationic charge of at least 1 meq/g, and the lotion pH being from about 3.5 to about 5.5. The lotion composition may be incorporated along with a substrate into a wet wipe.

### FIELD

The present disclosure includes lotion compositions comprising soil capture polymers for use in cleansing of organic bodily soils.

### BACKGROUND

A variety of cleansing products for household soils are known in the art, both in the form of lotion compositions that can be sprayed or otherwise deposited onto a surface and then absorbed by a substrate, and in the form of wet wipes that can be directly used to cleanse a surface. Some of these lotion compositions make use of soil capture polymers to flocculate the household soil and make it easier to capture and remove (for example, see U.S. application Ser. Nos. 13/598,782 and 14/548,614). However, none of these address the need of removing organic bodily soils, which are typically different in terms of chemical and physical properties (e.g. protein/fat/mucous content, particulate size, charge) from household soil.

Cleaning of any type of soil off of a surface may be accomplished by spraying or otherwise depositing a lotion composition onto the surface and then using a substrate to “wipe up” the composition-soil mixture, or by impregnating a substrate with a lotion composition to form a “wet wipe” and using said wet wipe to cleanse the surface. Lotion compositions or wet wipes comprising them may also be useful for delivering functional materials to a surface, such as to skin. For example, wet wipes may deliver materials that provide skin benefits, such as softening and/or moisturizing the skin, or protection from or treatment of diaper rash and other skin ailments such as eczema. The lotion compositions may also protect the skin from irritants present in bodily fluids like urine and bowel movement. These additional benefits may be provided while the lotion compositions or wet wipes enable the removal of soil.

Wet wipes are constructed from porous or absorbent sheets impregnated with a lotion composition, and they are sold and stored in an air-tight container or wrapper to prevent the sheets drying out. Wipes may be used with feminine health and adult incontinence products, but a major proportion of the wipes intended for the cleansing of human skin are wet wipes designed for use with infants and young children. Wet wipes are required to be effective at cleaning while at the same time being very gentle and mild on the skin of the baby.

Wipes may predominantly rely on physical mechanisms to soils within the open structure of a substrate, often with the aid of surfactants or solvents in a lotion composition incorporated into or onto the wipe. The soil capture polymer technology of the present invention involves lotion compositions comprising long-chain polymers, such as polyacrylamide-based polymers or quaternary vinyl imidazole (QVI) chemistries that are optimized to flocculate organic bodily soils and enable better capture of this soil type via the fibrous substrate.

### SUMMARY

A lotion composition comprises a soil capture polymer, the soil capture polymer having a number average molecular weight of at least about 1,000,000 and a cationic charge of at least about 1 meq/g, and the lotion pH being from about 3.5 to about 5.5. The lotion composition may be impregnated into a substrate to form a wet wipe.

### DETAILED DESCRIPTION

The following definitions may be useful in understanding the present disclosure:

As used herein, the term “lotion composition” means an aqueous (substantially water-based) non-emulsion or emulsion formulation comprising one or more components such as surfactants, rheology modifiers, preservatives, buffering agents, emollients, perfumes, skin benefiting ingredients, and soil capture polymers.

“Wet wipe” as used herein means any type of substrate to which a lotion composition has been applied at a ratio of grams lotion composition to grams substrate of 0.5 to 6.0.

“Loading” as used herein means the process of applying a lotion composition to a substrate to form a wet wipe. A “loaded” substrate is associated with a lotion composition.

“Soil” as used herein means matter that is extraneous to a surface being cleaned.

“Organic bodily soil” as used herein refers to bodily exudates such as feces, menses, urine, vomitus, mucus, and the like. Such exudates are often negatively charged.

“Cationic monomeric unit” as used herein means a monomeric unit that exhibits a net positive charge at a pH of 3.5 to 5.5 and/or is identified as a cationic monomeric unit herein. A cationic monomeric unit may be derived from a cationic monomer. A cationic monomeric unit is generally associated with one or more anions such as a chloride ion, a bromide ion, a sulfonate group and/or a methyl sulfate group.

“Cationic monomer” as used herein means a monomer that exhibits a net positive charge at a pH of 3.5 to 5.5 and/or is identified as a cationic monomer herein. A cationic monomer is generally associated with one or more anions such as a chloride ion, a bromide ion, a sulfonate group and/or a methyl sulfate group.

“Monomeric unit” as used herein means a constituent unit (sometimes referred to as a structural unit) of a polymer.

“Nonionic monomeric unit” as used herein means a monomeric unit that exhibits no net charge at a pH of 3.5 to 5.5 and/or is identified as a nonionic monomeric unit herein. A nonionic monomeric unit may be derived from a nonionic monomer.

“Nonionic monomer” as used herein means a monomer that exhibits no net charge at a pH of 3.5 to 5.5 and/or is identified as a nonionic monomer herein.

The term “hydrophilic coating” as used herein means a chemical treatment applied to a substrate to cause the substrate to become hydrophilic or more hydrophilic.

The term “hydrophilic” as used herein refers to a substrate or composition having a contact angle less than or equal to 90° according to The American Chemical Society Publication “Contact Angle, Wettability, and Adhesion,” edited by Robert F. Gould and copyrighted in 1964.

The term “hydrophobic coating” as used herein means a chemical treatment applied to a substrate to cause the substrate to become hydrophobic or more hydrophobic.

The term “hydrophobic” as used herein refers to a substrate or composition having a contact angle greater than or equal to 90° according to The American Chemical Society Publication “Contact Angle, Wettability, and Adhesion,” edited by Robert F. Gould and copyrighted in 1964.

“Number average molecular weight” as used herein means the number average molecular weight Mₙ as determined using gel permeation chromatography according to the Molecular Weight Test Method disclosed herein.

Weight average molecular weight” as used herein means the weight average molecular weight Mw as determined using gel permeation chromatography according to the protocol found in Colloids and Surfaces A. Physico Chemical & Engineering Aspects, Vol. 162, 2000, pg. 107-121.

“Substrate” as used herein means a fibrous structure made from an assembly of continuous fibers, coextruded fibers, non-continuous fibers and combinations thereof, without weaving or knitting, by processes such as spunbonding, carding, meltblowing, airlaying, wetlaying, coforming, or other such processes known in the art for such purposes. The terms “substrate” and “nonwoven” may be used interchangeably. A “substrate” is primarily two dimensional (i.e. in an XY plane) and has a thickness (in a Z direction) that is relatively small (i.e. 1/10 or less) in comparison to the substrate's length (in an X direction) and width (in a Y direction). Non-limiting examples of substrates include a web, layer or layers or fibrous materials, nonwovens, films and foils such as polymeric films or metallic foils. These materials may be used alone or may comprise two or more layers joined together.

“Q. S.” as used herein means “quantum sufficit” and is a sufficient percentage of water added to the lotion composition to bring the overall composition to 100%.

As used herein, percentages are given as the weight of the component to the total weight of the lotion composition, unless otherwise indicated. Percentages reflect 100% active component material. For example, if a component is available in a dispersion at a concentration of 50% component to dispersion, by weight, twice as much of the dispersion, by weight, would be added to the lotion composition to provide the equivalent of 100% active component.

Values disclosed herein as ends of ranges are not to be understood as being strictly limited to the exact numerical values recited. Instead, unless otherwise specified, each numerical range is intended to mean both the recited values and any integers within the range. For example a range disclosed as “1 to 10” is intended to mean “1, 2, 3, 4, 5, 6, 7, 8, 9, and 10.”

While the present disclosure references the use of a sprayed/deposited lotion composition or a wet wipe comprising a lotion composition for cleaning soils, it is to be appreciated that the lotion composition could be used in a variety of ways to achieve cleaning of the soil.

### Soil Capture Polymers

The lotion compositions of the present invention comprise soil capture polymers that may flocculate organic bodily soils, which can allow better capture by a substrate.

In one example of the present invention, a fibrous structure, for example an absorbent fibrous structure, comprises a soil capture polymer. In order to be effective, it is beneficial for a soil capture polymer to be able to flocculate soils. Once the soils are flocculated, it allows easier capture of the soils in a fibrous structure. It has been found that soils may be best flocculated and captured by fibrous structures when the fibrous structure includes a lotion composition comprising a polymer with a very high number average molecular weight. For household soils, the soils are best captured when the polymer has a low charge density and an overall neutral charge. But for organic bodily soils, such as are most relevant for baby wipes, capture is surprisingly effective with soil capture polymers that have a very high number average molecular weight and a very high level of overall charged moieties, especially an excess of cationic charge. The soil capture polymers may include materials that have a minimum number average molecular weight (Mₙ) of about 1,000,000 and a minimum cationic charge of about 1 meq/g.

A soil capture polymer as described herein provides enhanced benefits in capturing soil. Such soil capture polymers can be used singularly or in combination with other components to form a lotion composition. Soil capture polymers may include several monomeric units, so they may be referred to as a copolymer rather than a homopolymer, which consists of a single type of monomeric unit. The polymers of the present disclosure may be a terpolymer (3 different monomeric units). The polymers of the present disclosure may be a random copolymer. In one example, a polymer of the present disclosure may be water-soluble and/or water-dispersible, which means that the polymer does not, over at least a certain pH and concentration range, form a two-phase composition in water at 23° C.±2.2° C.

In one example, the soil capture polymers of the present invention exhibit a Number Average Molecular Weight of at least about 1,000,000 g/mol. In other embodiments, the soil capture polymer may have a Mₙ of at least about 1,500,000, in some cases at least about 2,000,000; 2,500,000; 3,000,000, 4,000,000; or 5,000,000. While the Mₙ of the soil capture polymer in theory may not be too high, the soil capture polymers may have a Mₙ of no more than about 10,000,000, in some cases, about 9,000,000; 8,000,000; 7,000,000; 6,000,000; 5,000,000; or 4,000,000. The range of Mₙ for the soil capture polymer may be any combination of lower limit to upper limit described herein. In some embodiments, the Mₙ of the soil capture polymer may be from about 1,000,000 to about 3,000,000; from about 1,500,000 to about 3,500,000, from about 2,000,000 to about 5,000,000, or from about 2,500,000 to about 5,000,000.

In yet another example, the polymers of the present invention exhibit a charge density (at pH 4.5) of at least about 1.0 meq/g and/or from about 0.8; 0.9; 1.0, 1.1; 1.2; 1.3; 1.4; 1.5; 1.6; or 1.7 meq/g to about 1.1; 1.2; 1.3; 1.4; 1.5; 1.6; 1.7; 1.8; 1.9; 2.0; 3.0; 4.0; 5.0; 6.0; 7.0; 8.0; 9.0; or 10.0 meq/g; and any combination of lower limit to upper limit described herein, as measured according to the Charge Density Test Method described herein. In still another example, the polymers of the present invention may exhibit a charge density of from about 1.0 meq/g to about 6 meq/g; from about 1.5 meq/g to about 6.0 meq/g; or from about 1.7 meq/g to about 5 meq/g, as measured according to the Charge Density Test Method described herein.

In one example, the soil capture polymers may have a maximum value of percent back-scattering at 2 minutes of at most about 12%, as measured using the ABM Flocculation and Settling Test Method described herein. In some embodiments, the soil capture polymer may have a maximum value of percent back-scattering at 2 minutes of about 7% to about 12%; or about 7% to about 10%; in some embodiments from about 7%; 7.5%; 8%; 8.5%; 9%; 9.5%; 10%; or 11% to about 8%; 9%; 10%; 11%; or 12%, or any combination of lower limit to upper limit described herein. This percent back-scattering value indicates the flocculation ability of the soil capture polymers.

Particular polymers that can provide the soil capture capability may include, for example, a long-chain polymer such as a polyacrylamide-based polymer. Appropriate soil capture polymers include QVI (quaternary vinylimidazole) and VI (vinylimidazole) based polymers. In some embodiments, a polymer of the present invention comprises monomeric units such as those listed below:

a. Nonionic Monomeric Units

The nonionic monomeric units may be selected from the group consisting of: nonionic hydrophilic monomeric units, nonionic hydrophobic monomeric units, and mixtures thereof.

Non-limiting examples of nonionic hydrophilic monomeric units suitable for the present invention include nonionic hydrophilic monomeric units derived from nonionic hydrophilic monomers selected from the group consisting of: hydroxyalkyl esters of α,β-ethylenically unsaturated acids, such as hydroxyethyl or hydroxypropyl acrylates and methacrylates, glyceryl monomethacrylate, α,β-ethylenically unsaturated amides such as acrylamide, N,N-dimethylacrylamide, N,N-dimethylmethacrylamide, N-methylolacrylamide, α,β-ethylenically unsaturated monomers bearing a water-soluble polyoxyalkylene segment of the poly(ethylene oxide) type, such as poly(ethylene oxide) α-methacrylates (Bisomer S20W, S10W, etc., from Laporte) or α,ω-dimethacrylates, Sipomer BEM from Rhodia (ω-behenyl polyoxyethylene methacrylate), Sipomer SEM-25 from Rhodia (ω-tristyrylphenyl polyoxyethylene methacrylate), α,β-ethylenically unsaturated monomers which are precursors of hydrophilic units or segments, such as vinyl acetate, which, once polymerized, can be hydrolyzed in order to give rise to vinyl alcohol units or polyvinyl alcohol segments, vinylpyrrolidones, α,β-ethylenically unsaturated monomers of the ureido type, and in particular 2-imidazolidinone-ethyl methacrylamide (Sipomer WAM II from Rhodia), and mixtures thereof. In one example, the nonionic hydrophilic monomeric unit is derived from acrylamide.

Non-limiting examples of nonionic hydrophobic monomeric units suitable for the present invention include nonionic hydrophobic monomeric units derived from nonionic hydrophobic monomers selected from the group consisting of: vinylaromatic monomers such as styrene, alpha-methylstyrene, vinyltoluene, vinyl halides or vinylidene halides, such as vinyl chloride, vinylidene chloride, C₁-C₁₂ alkylesters of α,β-monoethylenically unsaturated acids such as methyl, ethyl or butyl acrylates and methacrylates, 2-ethylhexyl acrylate, vinyl esters or allyl esters of saturated carboxylic acids, such as vinyl or allyl acetates, propionates, versatates, stearates, α,β-monoethylenically unsaturated nitriles containing from 3 to 12 carbon atoms, such as acrylonitrile, methacrylonitrile, α-olefins such as ethylene, conjugated dienes, such as butadiene, isoprene, chloroprene, and mixtures thereof.

b. Cationic Monomeric Units

Non-limiting examples of cationic monomeric units suitable for the present invention include amine containing monomeric units derived from monomers selected from the group consisting of: N,N-(dialkylamino-w-alkyl)amides of α,β-monoethylenically unsaturated carboxylic acids, such as N,N-dimethylaminomethyl-acrylamide or -methacrylamide, 2-(N,N-dimethylamino)ethylacrylamide or -methacrylamide, 3-(N,N-dimethylamino)propylacrylamide or -methacrylamide, and 4-(N,N-dimethylamino)butylacrylamide or -methacrylamide, α,β-monoethylenically unsaturated amino esters such as 2-(dimethylamino)ethyl acrylate (DMAA), 2-(dimethylamino)ethyl methacrylate (DMAM), 3-(dimethylamino)propyl methacrylate, 2-(tert-butylamino)ethyl methacrylate, 2-(dipentylamino)ethyl methacrylate, and 2(diethylamino)ethyl methacrylate, vinylpyridines, vinylamine, vinylimidazolines, monomers that are precursors of amine functions such as N-vinylformamide, N-vinylacetamide, which give rise to primary amine functions by simple acid or base hydrolysis, acryloyl- or acryloyloxyammonium monomers such as trimethylammonium propyl methacrylate chloride, trimethylammoniumethylacrylamide or -methacrylamide chloride or bromide, trimethylammonium butylacrylamide or -methacrylamide methyl sulfate, trimethylammonium propylmethacrylamide methyl sulfate, (3-methacrylamidopropyl)trimethylammonium chloride (MAPTAC), (3-methacrylamidopropyl)trimethylammonium methyl sulphate (MAPTA-MES), (3-acrylamidopropyl)trimethylammonium chloride (APTAC), methacryloyloxyethyl-trimethylammonium chloride (METAC) or methyl sulfate, and acryloyloxyethyltrimethylammonium chloride (AETAC); 1-ethyl-2-vinylpyridinium or 1-ethyl-4-vinylpyridinium bromide, chloride or methyl sulfate; N,N-dialkyldiallylamine monomers such as N,N-dimethyldiallylammonium chloride (DADMAC); polyquaternary monomers such as dimethylaminopropylmethacrylamide chloride and N-(3-chloro-2-hydroxypropyl)trimethylammonium (DIQUAT or DQ) and 2-hydroxy-N1-(3-(2((3-methacrylamidopropyl)dimethylammino)-acetamido)propyl)-N1,N1,N3,N3,N3-pentamethylpropane-1,3-diaminium chloride (TRIQUAT or TQ), and mixtures thereof. In one example, the cationic monomeric unit comprises a quaternary ammonium monomeric unit, for example a monoquaternary ammonium monomeric unit, a diquaternary ammonium monomeric unit and a triquaternary monomeric unit. In one example, the cationic monomeric unit is derived from MAPTAC. In another example, the cationic monomeric unit is derived from DADMAC. In still another example, the cationic monomeric unit is derived from TQ.

In one example, the non-ionic monomers are selected from acrylamide derivatives from the group consisting of: acrylamide, mono-alkyl substituted acrylamide, symmetrical or asymmetrical, di-N-alkyl substituted acrylamide derivatives, methacrylamide, mono-alkyl substituted methacrylamide, symmetrical or asymmetrical, di-N-alkyl substituted methacrylamide derivatives and mixtures thereof.

In another example, the acrylamide derivatives of the present invention are selected from the group consisting of: N,N-dimethylacrylamide (NDMAAM), acrylamide, methyl acrylamide, ethylacrylamide, N,N-diethylacrylamide, methacrylamide, N,N-dimethyl methacrylamide, and mixtures thereof.

Further examples of cationic monomeric units suitable for the present invention include cationic monomeric units derived from cationic monomers selected from the group consisting of: N,N-(dialkylamino-w-alkyl)amides of α,β-monoethylenically unsaturated carboxylic acids, such as N,N-dimethylaminomethylacrylamide or -methacrylamide, 2-(N,N-dimethylamino)ethylacrylamide or -methacrylamide, 3-(N,N-dimethylamino)propylacrylamide or -methacrylamide, and 4-(N,N-dimethylamino)butylacrylamide or -methacrylamide, α,β-monoethylenically unsaturated amino esters such as 2-(dimethylamino)ethyl acrylate (DMAA), 2-(dimethylamino)ethyl methacrylate (DMAM), 3-(dimethylamino)propyl methacrylate, 2-(tert-butylamino)ethyl methacrylate, 2-(dipentylamino)ethyl methacrylate, and 2(diethylamino)ethyl methacrylate, vinylpyridines, vinylamine, vinylimidazolines, monomers that are precursors of amine functions such as N-vinylformamide, N-vinylacetamide, which give rise to primary amine functions by simple acid or base hydrolysis, acryloyl- or acryloyloxyammonium monomers such as trimethylammonium propyl methacrylate chloride, trimethylammoniumethylacrylamide or -methacrylamide chloride or bromide, trimethylammonium butylacrylamide or -methacrylamide methyl sulfate, trimethylammonium propylmethacrylamide methyl sulfate, (3-methacrylamidopropyl)trimethylammonium chloride (MAPTAC), (3-methacrylamidopropyl)trimethylammonium methyl sulphate (MAPTA-MES), (3-acrylamidopropyl)trimethylammonium chloride (APTAC), methacryloyloxyethyl-trimethylammonium chloride or methyl sulfate, and acryloyloxyethyltrimethylammonium chloride; 1-ethyl-2-vinylpyridinium or 1-ethyl-4-vinylpyridinium bromide, chloride or methyl sulfate; N,N-dialkyldiallylamine monomers such as N,N-dimethyldiallylammonium chloride (DADMAC); polyquaternary monomers such as dimethylaminopropylmethacrylamide chloride and N-(3-chloro-2-hydroxypropyl)trimethylammonium (DIQUAT or DQ) and 2-hydroxy-N¹-(3-(2((3-methacrylamidopropyl)dimethylammino)-acetamido)propyl)-N¹,N¹,N³,N³,N³-pentamethylpropane-1,3-diaminium chloride (TRIQUAT or TQ), and mixtures thereof. In one example, the cationic monomeric unit comprises a quaternary ammonium monomeric unit, for example a monoquaternary ammonium monomeric unit, a diquaternary ammonium monomeric unit and a triquaternary monomeric unit. In one example, the cationic monomeric unit is derived from MAPTAC. In another example, the cationic monomeric unit is derived from DADMAC. In still another example, the cationic monomeric unit is derived from TQ.

In one example, the cationic monomeric units are derived from cationic monomers selected from the group consisting of: dimethylaminoethyl (meth)acrylate, dimethylaminopropyl (meth)acrylate, di-tert-butylaminoethyl (meth)acrylate, dimethylaminomethyl (meth)acrylamide, dimethylaminopropyl (meth)acrylamide, ethylenimine, vinylamine, 2-vinylpyridine, 4-vinylpyridine and vinyl imidazole, and mixtures thereof.

In another example, the cationic monomeric units are derived from cationic monomers selected from the group consisting of: trimethylammoniumethyl (meth)acrylate bromide, chloride or methyl sulfate, trimethylammoniumethyl (meth)acrylate bromide, chloride or methyl sulfate, trimethylammoniumethyl (meth)acrylate bromide, chloride or methyl sulfate, dimethylaminoethyl (meth)acrylate benzyl chloride, 4-benzoylbenzyl dimethylammoniumethyl (meth)acrylate bromide, chloride or methyl sulfate, trimethylammoniumethyl (meth)acrylamido bromide, chloride, or methyl sulfate, trimethylammonium propyl (meth)acrylamido braomide, chloride, or methyl sulfate, vinyl benzyl trimethylammonium bromide, chloride or methyl sulfate, diallyldimethyl ammonium chloride, 1-ethyl-2-vinylpyridinium bromide, chloride or methyl sulfate, 4-vinylpyridinium bromide, chloride or methyl sulfate, and mixtures thereof.

### Process for Making Polymers

The polymers of the present invention may be made by any suitable process known in the art. For example, the polymer may be made by radical polymerization.

The polymers of the present invention can be made by a wide variety of techniques, including bulk, solution, emulsion, or suspension polymerization. Polymerization methods and techniques for polymerization are described generally in Encyclopedia of Polymer Science and Technology, Interscience Publishers (New York), Vol. 7, pp. 361-431 (1967), and Kirk-Othmer Encyclopedia of Chemical Technology, 3rd edition, Vol 18, pp. 740-744, John Wiley & Sons (New York), 1982, both incorporated by reference herein. See also Sorenson, W. P. and Campbell, T. W., Preparative Methods of Polymer Chemistry. 2nd edition, Interscience Publishers (New York), 1968, pp. 248-251, incorporated by reference herein, for general reaction techniques suitable for the present invention. In one example, the polymers are made by free radical copolymerization, using water soluble initiators. Suitable free radical initiators include, but are not limited to, thermal initiators, redox couples, and photochemical initiators. Redox and photochemical initiators may be used for polymerization processes initiated at temperatures below about 30° C. (86° F.). Such initiators are described generally in Kirk-Othmer Encyclopedia of Chemical Technology, 3rd edition, John Wiley & Sons (New York), Vol. 13, pp. 355-373 (1981), incorporated by reference herein. Typical water soluble initiators that can provide radicals at 30° C. or below include redox couples, such as potassium persulfate/silver nitrate, and ascorbic acid/hydrogen peroxide. In one example, the method utilizes thermal initiators in polymerization processes conducted above 40° C. (104° F.). Water soluble initiators that can provide radicals at 40° C. (104° F.) or higher can be used. These include, but are not limited to, hydrogen peroxide, ammonium persulfate, and 2,2′-azobis(2-amidinopropane) dihydrochloride. In one example, water soluble starting monomers are polymerized in an aqueous alcohol solvent at 60° C. (140° F.) using 2,2′-azobis(2-amidinopropane) dihydrochloride as the initiator.

### Non-limiting Synthesis Examples Sample Preparation

### a. Synthesis of 3-methyl-1-vinyl-1H-imidazol-3-ium chloride (QVI-Cl)

Synthesis is performed using a Model Number 4572 Parr 1800 ml reactor constructed of T316 stainless steel equipped with magnetic drive stirring assembly that uses an electric motor for agitation with a stir shaft that has 2 each pitched blade impellers and a cooling coil to maintain the temperature from exceeding a programmed set point as monitored by Camille data acquisition and control system. To this reactor is added 107.1 g vinyl imidazole, 0.1151 g 4-methoxyphenol as a polymerization inhibitor, and 280.7 g acetonitrile. The reaction mixture is purged with nitrogen and then 218 g methyl chloride is added. Mechanical agitation is used throughout the reaction at 250 RPM. The reactor is heated and kept between 75° C. to 80° C. for 20 hours and then cooled to and held at 50° C. for 24 hours. The reaction is purged with nitrogen to remove excess methyl chloride and 350 g of chloroform is added to the sample mixture to aid in removal from the reaction vessel.

The resultant liquor is filtered to remove solid particulate and then concentrated to approximately 300 g using rotary evaporation. Then 75 mL of ethanol is added and the liquor and is filtered once more to remove solids. The liquor is then poured into 3500 mL of acetone with rapid stirring. The precipitate is filtered, then washed with 500 mL clean acetone. The powdery solid is retained and residual solvent is removed by vacuum evaporation. The solid is dissolved into water to a concentration of 64.2% on a mass active to mass solution basis.

### b. Poly(QVI-Cl)

To a 40 mL reaction vessel 23.36 g of 3-methyl-1-vinyl-1H-imidazol-3-ium chloride (64.2%) in water and an additional 5.64 g of water is added. To this 1.00 g of an initiator solution comprised of 0.0280 g 2,2′-azobis(2-methylpropionamidine) dihydrochloride [available from Sigma Aldrich, catalog #440914] and 4.6441 g water is added. The solution is sealed, sparged for 3 minutes under an inert gas such as argon, and then heated to a temperature of 55° C. for 72 hours. The resultant polymer gel is diluted to approximately 3% active with water to form a free flowing fluid. This fluid is poured into excess isopropanol and the polymer is precipitated into a gelatinous material. The polymer precipitate is rinsed with clean isopropanol and excess solvent removed by vacuum evaporation. The remaining polymer solids are dissolved back into water to the desired concentration.

### c. Poly(QVI-Cl-co-VI)

To 40 mL reaction vesselsQVI-Cl (64.2%), vinyl imidazole (VI) (available from Sigma Aldrich, catalog #235466), and water in the amounts listed in the Table 1 are added. Then 2.5 g of concentrated HCl available for EMD Chemicals as catalog number HX0603-4, is added to ensure the pH of the solution is at or below a pH value of 1. To this 1.00 g of an initiator solution comprised of 0.0280 g of 2,2′-azobis(2-methylpropionamidine) dihydrochloride [available from Sigma Aldrich, cat#440914] and 4.6441 g water is added. The solution is sealed, sparged for 3 minutes under an inert gas such as argon, and then heated to a temperature of 55° C. for 72 hours. The resultant polymer solution is diluted to approximately 5% active with water. The polymer solution is then poured into excess isopropanol to form a precipitate. The polymer precipitate is rinsed with clean isopropanol and excess solvent is removed by vacuum evaporation. The remaining polymer solids are dissolved back into water to the desired concentration.

TABLE 1

| | QVI-CI | | |
|---|---|---|---|
| | solution | | Water |
| Example | (g) | VI (g) | (g) |
| 95% QVI-CI 5% VI | 22.2017 | 0.7445 | 3.55 |
| 90% QVI-CI 10% VI | 21.0304 | 1.4980 | 3.97 |
| 85% QVI-CI 15% VI | 19.8631 | 2.2491 | 4.40 |

The soil capture polymers of the present invention may be incorporated into a lotion composition, which can then be applied to a substrate.

### Lotion Compositions

The lotion composition may comprise from about 0.01% to about 1.0% of at least one soil capture polymer. In some embodiments, the lotion composition may comprise from about 0.1% to about 0.5% of at least one soil capture polymer, or from about 0.1% to about 0.25% of at least one soil capture polymer, or from about 0.2% to about 0.25% of at least one soil capture polymer.

The lotion composition may comprise a preservative system. In some exemplary configurations, the preservative system may include a preservative enhancing agent and one or more preservatives. A preservative may be understood to be a chemical or natural compound or a combination of compounds reducing the growth of microorganisms, thus enabling a longer shelf life for a package of substrates (opened or not opened) as well as creating an environment with reduced growth of microorganisms when transferred to the skin during the wiping process.

Low pH buffering systems, such as a citrate-citric acid buffering system from a pH of about 3.5 to about 5.5, may also be employed as part of the preservative system. In some embodiments, the pH may be from about 3.5 to about 4.1 or from about 4.1 to about 4.7.

The lotion composition also includes a carrier such as water. The lotion composition may comprise greater than about 50%, greater than about 60%, greater than about 70%, greater than about 80%, greater than about 90%, greater than about 95%, greater than about 96%, greater than about 97%, greater than about 98%, greater than about 98.5%, greater than about 99%, or greater than about 99.5% by weight of water. In addition, the lotion composition may include various optional ingredients, such as surfactants, emollients, film-formers, preservatives, pH buffers, rheology modifiers and the like, such as described in U.S. Pat. Nos. 7,666,827; 7,005,557; 8,221,774; and U.S. Patent Application Publication No. 2011/0268777. For example, the lotion composition may comprise optional ingredients such as perfumes, aloe, and chamomile.

The preservative system may include one or more preservative enhancing agents. Exemplary preservative enhancing agents include sorbitan caprylate, glyceryl caprylate/caprate, or combinations thereof. An exemplary sorbitan caprylate is manufactured by Clariant under the designation VELSAN® SC. An exemplary glyceryl caprylate/caprate may be CremerCOOR® GC810, CremerCOOR® GCB, or IMWITOR® 742, all available from Peter Cremer, or CAPMUL® 708G, available from Abitec.

The lotion composition may comprise from about 0.05% to about 0.30% by weight of a preservative enhancing agent. In some embodiments, the lotion composition may comprise at most about 0.2% by weight of a preservative enhancing agent. The lotion composition may comprise one or more rheology modifiers. A rheology modifier may help to stabilize the lotion composition by reducing or preventing coalescence of droplets of the hydrophobic materials in the composition. Non-limiting examples of rheology modifiers include, but are not limited to, hydrocolloids, including natural gums. In general, the rheology modifiers in the present invention may be neutral or cationic in charge to avoid interaction with the cationic soil capture polymers. Rheology modifiers, when present in the lotion composition, may be present in the range of about 0.01% to about 0.25% by weight, or in the range of about 0.05% to about 0.18%, or about 0.06% by weight.

The preservative system of the lotion composition may comprise one or more preservative enhancing agents in combination with one or more preservatives. It has been found that a wet wipe having a lotion composition comprising a preservative enhancing agent and a preservative may have improved antimicrobial performance compared to a wet wipe having a lotion composition comprising a preservative without a preservative enhancing agent. As a result, lower concentrations of a preservative may be used in a lotion composition comprising a preservative enhancing agent than may be used when the lotion composition comprises a preservative without a preservative enhancing agent.

The lotion composition may include one or more preservatives. The preservative may include an organic acid or the salt thereof. Exemplary organic acids include benzoic acid or sorbic acid. Exemplary salts of organic acids include sodium benzoate and potassium sorbate, for example. The lotion composition may comprise from about 0.1% to about 0.3% of the exemplary organic acids or salts. In some embodiments, the lotion composition may comprise from about 0.18% to 0.24% of the exemplary organic acids or salts.

The preservative system of the lotion composition may include additional compounds, for example chelating agents, such as ethylenediamine tetraacetic acid (EDTA) and its salts, or diethylene triamine pentaacetic acid (DTPA).

An exemplary wet wipe may include a lotion composition comprising a preservative enhancing agent and a preservative. In an exemplary configuration, the lotion composition may comprise sorbitan caprylate and/or glyceryl caprylate/caprate and sodium benzoate. In a further exemplary configuration, the lotion composition may comprise sorbitan caprylate and/or glyceryl caprylate/caprate, sodium benzoate, a chelating agent, and a citrate-citric acid buffering system at a pH of about 3.5 to about 5.5.

The lotion composition comprising a preservative enhancing agent and a preservative may be incorporated into a substrate at a ratio of about 2.0 g lotion composition/g substrate to a ratio of about 6.0 g lotion composition/g substrate. In some embodiments, a wet wipe comprising a substrate may comprise or be impregnated with the lotion composition at a ratio of about 3.0 g lotion composition/g substrate to a ratio of about 5.0 g lotion composition/g substrate.

Additional ingredients may be added to the lotion composition. The lotion composition may generally comprise any of the following ingredients: emollients, surfactants, rheology modifiers, or other adjunct ingredients such as texturizers, colorants, opacifying agents, soothing agents and medically active ingredients, such as healing actives and skin protectants. It is to be noted that some ingredient compounds can have a multiple function and that all compounds are not necessarily present in the lotion composition.

An emollient may include silicone oils, functionalized silicone oils, hydrocarbon oils, fatty alcohols, fatty alcohol ethers, fatty acids, esters of monobasic and/or dibasic and/or tribasic and/or polybasic carboxylic acids with mono and polyhydric alcohols, polyoxyethylenes, polyoxypropylenes, mixtures of polyoxyethylene and polyoxypropylene ethers of fatty alcohols, and mixtures thereof. The emollients may be either saturated or unsaturated, have an aliphatic character and be straight or branched chained or contain alicyclic or aromatic rings.

The lotion composition may include one or more surfactants. The surfactant can be an individual surfactant or a mixture of surfactants. The surfactant may be a polymeric surfactant or a non-polymeric one. The surfactant or combinations of surfactants may be mild, which means that the surfactants provide sufficient cleaning or detersive benefits but do not overly dry or otherwise harm or damage the skin. In general, surfactants in the present invention will typically not be anionic, due to the cationic nature of the soil capture polymers. The surfactant, when present in the lotion composition, may be present in an amount ranging from about 0.05% to about 1% by weight of the lotion composition.

In some exemplary configurations, the surfactant may comprise PEG-40 Hydrogenated Castor Oil, such as EMULSOGEN® HCW049 manufactured by Clariant.

### SUBSTRATE

The lotion composition of the present disclosure may be loaded onto a substrate to form a wet wipe. The substrate may be a nonwoven material. The nonwoven material may comprise one or more layers of such fibrous assemblies, wherein each layer may include continuous fibers, coextruded fibers, non-continuous fibers and combinations thereof.

The fibers of the substrate may be comprised of any natural, cellulosic, and/or wholly synthetic material. Examples of natural fibers may include cellulosic natural fibers, such as fibers from hardwood sources, softwood sources, or other non-wood plants. The natural fibers may comprise cellulose, starch and combinations thereof. Non-limiting examples of suitable cellulosic natural fibers include wood pulp, typical northern softwood Kraft, typical southern softwood Kraft, typical CTMP, typical deinked, corn pulp, acacia, eucalyptus, aspen, reed pulp, birch, maple, radiata pine and combinations thereof. Other sources of natural fibers from plants include albardine, esparto, wheat, rice, corn, sugar cane, papyrus, jute, reed, sabia, raphia, bamboo, sidal, kenaf, abaca, sunn, rayon (also known as viscose), lyocell, cotton, hemp, flax, ramie and combinations thereof. Yet other natural fibers may include fibers from other natural non-plant sources, such as, down, feathers, silk, cotton and combinations thereof. The natural fibers may be treated or otherwise modified mechanically or chemically to provide desired characteristics or may be in a form that is generally similar to the form in which they can be found in nature. Mechanical and/or chemical manipulation of natural fibers does not exclude them from what are considered natural fibers with respect to the development described herein.

The synthetic fibers can be any material, such as those selected from the group consisting of polyesters (e.g., polyethylene terephthalate), polyolefins, polypropylenes, polyethylenes, polyethers, polyamides, polyesteramides, polyvinylalcohols, polyhydroxyalkanoates, polysaccharides, and combinations thereof. Further, the synthetic fibers can be a single component (i.e., single synthetic material or mixture makes up entire fiber), bi-component (i.e., the fiber is divided into regions, the regions including two or more different synthetic materials or mixtures thereof and may include coextruded fibers and core and sheath fibers) and combinations thereof. Bicomponent fibers can be used as a component fiber of the structure, and/or they may be present to act as a binder for the other fibers present in the fibrous structure. Any or all of the synthetic fibers may be treated before, during, or after manufacture to change any desired properties of the fibers. The substrate may comprise hydrophilic fibers, hydrophobic fibers, or a combination thereof.

The substrate may comprise various percentages of natural and/or synthetic fibers. For example, in some exemplary configurations, the substrate may comprise 100% synthetic fibers. In another exemplary configuration, the substrate may comprise natural and synthetic fibers. For example, the substrate may comprise from about 0% to about 90% natural fibers, with the balance comprising synthetic fibers. The substrate may be comprised of 10%, 20%, 30%, 40%, 50%, 60%, 70%, or 80% natural fibers. In some embodiments, the substrate may comprise at least about 15% cellulose, and in other embodiments, at least about 40% cellulose.

In certain configurations, it may be desirable to have particular combinations of fibers to provide desired characteristics. For example, it may be desirable to have fibers of certain lengths, widths, coarseness or other characteristics combined in certain layers, or separate from each other. The fibers may be of virtually any size and may have an average length from about 1 mm to about 60 mm. Average fiber length refers to the length of the individual fibers if straightened out. The fibers may have an average fiber width of greater than about 5 micrometers. The fibers may have an average fiber width of from about 5 micrometers to about 50 micrometers. The fibers may have a coarseness of greater than about 5 mg/100 m. The fibers may have a coarseness of from about 5 mg/100 m to about 75 mg/100 m.

The fibers may be circular in cross-section, dog-bone shape, delta (i.e., triangular cross section), trilobal, ribbon, or other shapes typically produced as staple fibers. Likewise, the fibers can be conjugate fibers such as bicomponent fibers. The fibers may be crimped and may have a finish, such as a lubricant, applied.

The substrate materials may also be treated to improve the softness and texture thereof. The substrate may be subjected to various treatments, such as physical treatment, hydro-molding, hydro-embossing, and ring rolling, as described in U.S. Pat. No. 5,143,679; structural elongation, as described in U.S. Pat. No. 5,518,801; consolidation, as described in U.S. Pat. Nos. 5,914,084; 6,114,263; 6,129,801 and 6,383,431; stretch aperturing, as described in U.S. Pat. Nos. 5,628,097; 5,658,639; and 5,916,661; differential elongation, as described in U.S. Pat. No. 7,037,569, and other solid state formation technologies as described in U.S. Pat. No. 7,553,532 and U.S. Pat. No. 7,410,683; zone activation, and the like; chemical treatment, such as rendering part or all of the substrate hydrophobic, and/or hydrophilic, and the like; thermal treatment, such as thermal-embossing, softening of fibers by heating, thermal bonding and the like; and combinations thereof.

Without wishing to be bound by theory, it is believed that a textured substrate may further enable the ease of removal of soils by improving the ability to grip or otherwise lift the soils from the surface during cleansing. Any one of a number of texture elements may be useful in improving the ability to grip or otherwise lift the soil from the surface during cleansing such as continuous hydro-molded elements, hollow molded element, solid molded elements, circles, squares, rectangles, ovals, ellipses, irregular circles, swirls, curly cues, cross hatches, pebbles, lined circles, linked irregular circles, half circles, wavy lines, bubble lines, puzzles, leaves, outlined leaves, plates, connected circles, changing curves, dots, honeycombs, and the like, and combinations thereof. The texture elements may be hollow elements. The texture elements may be connected to each other. The texture elements may overlap each other.

The substrate may have a basis weight between about 15, 30, 40, or 45 grams/m² and about 65, 75, 85, 95, or 100 grams/m². A suitable substrate may be a carded nonwoven comprising a 40/60 blend of viscose fibers and polypropylene fibers having a basis weight of 58 grams/m² as available from Suominen of Tampere, Finland as FIBRELLA® 3160. FIBRELLA® 3160 is a 58 grams/m² nonwoven web comprising 60% by weight of 1.5 denier polypropylene fibers and 40% by weight of 1.5 denier viscose fibers. Another suitable material may be FIBRELLA® 3100 which is a 62 grams/m² nonwoven web comprising 50% by weight of 1.5 denier polypropylene fibers and 50% by weight of 1.5 denier viscose fibers. In both of these commercially available fibrous webs, the average fiber length is about 38 mm. Another suitable material for use as a substrate may be SAWATEX® 2642 as available from Sandler AG of Schwarzenbach/Salle, Germany. Yet another suitable material for use as a substrate may have a basis weight of from about 50 grams/m² to about 60 grams/m² and have a 20/80 blend of viscose fibers and polypropylene fibers. The substrate may also be a 60/40 blend of pulp and viscose fibers. Exemplary nonwoven substrates are described in U.S. Patent Publication 2012/066852 and U.S. Patent Publication U.S. 2011/244199.

In some configurations, the surface of the substrate may be essentially flat. In other configurations, the surface of the substrate may optionally contain raised and/or lowered portions. The raised and/or lowered portions can be in the form of logos, indicia, trademarks, geometric patterns, and/or images of the surfaces that the substrate is intended to clean (i.e., infant's body, face, etc.). The raised and/or lowered portions may be randomly arranged on the surface of the substrate or be in a repetitive pattern of some form.

In yet other configurations, the substrate may be biodegradable. For example, the substrate could be made from a biodegradable material such as a polyesteramide, or a high wet strength cellulose. In some exemplary configurations, the substrate may be dispersible.

### Article of Commerce

In one embodiment, an article of commerce may be provided. The article of commerce may comprise a container and at least one wet wipe as described herein.

Containers may include, but are not limited to, PET tubs, flow wrap pouches, and other packaging known in the art as suitable for nonwoven articles. Additionally, the container may also be manufactured to facilitate removal of individual wet wipes.

The container may be made of any suitable material or materials and can be manufactured in any suitable manner. For example, the container can be made of polystyrene, polypropylene, PET, POET, polyethylene, polyester, polyvinyl alcohol, or the like. The containers may also be made of a mixture of the above materials. The containers may be manufactured by, for example, a vacuum molding process or an injection molding process, or any suitable process.

Additional information on containers, as well as additional optional components for containers, including, but not limited to: container bodies, lids, container features, such as, but not limited to, attachment of lids, hinges, zippers, securing aids, and the like, can be found in U.S. Pat. Nos. Des. 451,279; Des. 437,686; Des. 443,508; Des 443,451; Des 421,901; Des 421,902; Des 416,794; Des 414,637; Des 445,329; 3,982,659; 3,967,756; 3,986,479; 3,994,417; 6,269,970; 5,785,179; 5,366,104; 5,322,178; 5,050,737; 4,971,220; 6,296,144; 6,315,114; 4,840,270; 4,471,881; 5,647,506; 6,401,968; 6,269,969; 6,412,634; 5,791,465; 6,092,690; U.S. Patent Application Publication No. 2002/0064323 published on May 30, 2002, issued to Chin; and WO 00/27268 published on May 18, 2000 and assigned to The Procter & Gamble Company; WO 02/14172 published on Feb. 21, 2002 and assigned to The Procter & Gamble Company; and WO 99/55213 published on Nov. 4, 1999 and assigned to The Procter & Gamble Company.

In addition, the lotion compositions of the present invention may be put into a bottle or similar container, so that the consumer may choose whatever substrate is desired to apply the lotion composition onto for further use.

### Test Methods

### Molecular Weight

Polymer molecular mass is determined by GPC SEC/MALS. The HPLC is a Waters Alliance 2695 HPLC with an auto injector equipped with series of TSKgel PWxl-CP cationic modified columns available from Tosoh Biosciences LLC, 3604 Horizon Drive, King of Prussia, Pa., 19406. The column series consists of part numbers 21876, 21875, 21874, and 21873 respective to flow. The flow rate is 1.0 mL/min and the mobile phase is a 0.5 molar sodium acetate dissolved in 3 parts water to 1 part acetonitrile by volume. The detectors are Wyatt 5 Dawn EOS Light scattering detector calibrated with toluene and normalized using 25K dextran in mobile phase and a Wyatt Optilab rEX refractive index detector at 30° C.

Samples for analysis are prepared at a known concentration in the range of 1 to 5 mg/mL. Samples are filtered using 0.2 μm polypropylene membrane filters. The injection volume is 100 μL. The data are collected and analyzed using ASTRA 5.3.4.14. Values for do/dc are calculated from the RI trace assuming 100% mass recovery. Number average molecular weight, weight average molecular weight, and polydispersity index are calculated and reported.

### Charge Density

The charge density of a soil capture polymer is determined by using a Mutek PCD-04 Particle Charge Detector available from BTG or equivalent instrumentation.
1. Make a 0.1% polymer solution in water and adjust the pH to 4±0.25. Solution pH is adjusted prior to final dilution as charge density of many additives is dependent upon solution pH.
2. Place 20 mL of DI water into the PCD measuring cell and add 1 mL of polymer solution, then insert piston.
3. Put the measuring cell with piston and sample in the PCD, the electrodes are facing the rear. Slide the cell along the guide until it touches the rear.
4. Pull piston upwards and turn it counter-clock-wise to lock the piston in place.
5. Use the anionic titrant 0.001N potassium polyvinyl sulfate, herein PVSK, available from BTG.
6. An automatic titrator available from BTG is utilized. Set the titrator to rinse the tubing by dispensing 10 mL ensuring that all air bubbles have been purged.
7. Switch on the motor. The streaming potential is shown on the touch panel. Wait 2 minutes until the signal is stable.
8. Place tubing tip below the surface of the sample and start titration. The automatic titrator is set to stop automatically when the potential reaches 0 mV.
9. Record consumption of titrant.
10. Calculate charge demand.

$$Charge demand ( meq \text{/} g ) = ( \frac{V titrant used ( L ) \times Conc . of titrant in Normalitiy ( eq \text{/} L )}{Wt . solids of the sample or its active substance ( g )} ) \times 1000$$

Lotion Expression from Loaded Wipes

Expressed lotion compositions are prepared by inserting the entire wipe stack of a non-expired marketed product into a pre-cleaned press capable of exerting about 80 psi downward force on the stack. Ideally, the lower plate of the press contains a channel into which the expressed lotion may collect, and a hole through which the expressed lotion may flow into a clean storage container. An example of a suitable storage container is Catalog #83008-666 as available from VWR Scientific of West Chester, Pa. All expressed lotions are stored at room temperature prior to use.

### ABM Flocculation and Settling

The ABM (Artificial Bowel Movement) Flocculation and Settling Test Method is used to measure the Backscattering Value of a lotion composition comprising a soil capture polymer.

A Turbiscan™ LAB Thermo (“instrument”) available from Formulaction SA (10 impasse Borde-Basse—31240 I'Union—France) or equivalent instrument which measures backscattered light (Backscattering Value) is used for testing the ABM flocculation and settling.

The instrument has an electro luminescent diode in the near infrared (λair=880 nm).

The instrument has two synchronous optical detectors, one which receives transmitted light at 180° from the incident light and one which receives backscattered light at 45° from the incident light.

The instrument has specially designed Sample Cells (“Sample Cell”) that are flat bottomed glass cells (external diameter 27.5 mm, height 70 mm) with modified polycarbonate screwed top cap and butyl/Teflon sealing ring. Maximum volume within the Sample Cell is 22 mL. The materials to be tested must be inert in contact with glass and Teflon. The instrument scans the bottom 55 mm of the height of the Sample Cell, taking a measurement every 40 μm.

The incident light should hit the center of the Sample Cell so that it passes through 27.5 mm of material being tested.

The light beam which is 40 μm in size should pass through the material being tested for 0.1 seconds.

The instrument should be calibrated according to the manufacturer's instructions.

a. Soil

ABM (artificial bowel movement) is used for testing (“Soil”). See Making of ABM below.

b. Sample Cell Preparation

An empty, clean, Sample Cell specifically designed for the instrument is inspected to ensure no smudges or residues are present, and then handled with only gloved (nitrile examination gloves or equivalent) hands. If the Sample Cell is not empty, clean, smudge-free, residue-free, damage-free, then discard and get a new Sample Cell for use.

The Sample Cell is labeled with the specimen name on the cap, so that it will not interfere with the measurement, and tared.

1.0 g±0.1 g of the Soil is weighed (Weight_{Soil}) into the Sample Cell. The Sample Cell containing the Soil is re-tared.

Deionized water, 20.0 mL±0.2 mL, is added slowly to the Sample Cell using a suitable dispenser.

The Sample Cell containing the deionized water/Soil mixture is re-weighed to within ±0.1 mg (Weight_{Water}).

The cap is then placed on Sample Cell. After ensuring the Sample Cell is capped, the deionized water/Soil mixture is mixed for 5 seconds±1 second at 3200 rpm (max speed) and an amplitude of 0.358 cm using a vortexer (Vortex Genie 2 or equivalent) to ensure the Soil is suspended in the deionized water within the Sample Cell.

The Sample Cell's cap is then removed and 1 mL of 0.5% Soil Capture Polymer solution to be tested (“Test Sample”) is immediately added to the Sample Cell using a syringe.

The Sample Cell's cap is then immediately placed back onto the Sample Cell and the deionized water/Soil/Test Sample is immediately mixed for 5 seconds±1 second at 3200 rpm (max speed) and an amplitude of 0.358 cm using a vortexer (Vortex Genie 2 or equivalent) to ensure the Soil and the Test Sample are suspended in the deionized water within the Sample Cell.

The Sample Cell is then immediately [if this process (adding ABM to inserting Sample Cell into Turbiscan took more than 1 minute) then throw out and re-do the test] placed into the Instrument and the measurement is taken according to the Dynamic Test Sample Measurement Procedures as follows.

c. Dynamic Test Sample Measurement Procedure
1. Prior to Sample Cell Preparation (Step b above), turn on the Instrument and allow the system to warm up according to the manufacturer's instructions.
2. Dynamic Test Sample Measurements are taken as a scan up the Sample Cell (from the Sample Cell's bottom to a height along the Sample Cell of 55 mm) at each of: the initial time point (as soon as the Sample Cell is loaded into the test chamber of the Instrument) and a two minute time point.
3. The average percent backscatter (Backscattering Value) of a 10 mm height portion of the Sample Cell between 25 mm and 35 mm height from the bottom of the Sample Cell is recorded and reported. If a portion of the Test Sample is stuck to the glass between the 25 mm and 35 mm position, then discard and repeat the test for that Sample Cell.
4. Each condition is run in minimum triplicate and their average percent backscatter (Backscattering Value) from Step 3 above is then averaged to give the final Backscattering Value for that condition.

### Making of ABM (Artificial Bowel Movement)

a. Preparation of Dry Powder Mix (“Solid Premix”)

A solid premix was made according to the formula in Table 2, below. An IKA All basic grinder was used to grind the vegetables: dehydrated tomato dices (Harmony House or NorthBay); dehydrated spinach flakes (Harmony House or NorthBay); dehydrated cabbage (Harmony House or NorthBay); whole psyllium husk (available from Now Healthy Foods, sieved with 600 μm cutoff to collect greater than 600 μm particles and then ground to collect 250-300 μm particles) (alternatively available from Barry Farm as a powder that has to be sieved to collect 250-300 μm particles); palmitic acid (95% Alfa Aeser B20322); and calcium stearate (Alfa Aeser 39423). The vegetable flakes were added to the grinding bowl, filled to the mark (within the metal cup, not over-filled). The grinder was powered on for 5 seconds, stopped, and the powder was tapped 5 times. The grinder was again powered on and tapped 4 times (i.e., a total of 5 cycles of powering on and tapping). The ground powder was sieved by stacking a 600 μm opening sieve on top of a 300 μm opening sieve such that powders of 300 μm or less were collected. Any remaining powders that are larger than 300 μm are re-ground one time. Powders of 300 μm or less were collected. Next add food grade yeast powders commercially available as Provesta® 000 and Ohly® Auxoferm HCT (both commercially available from Ohly Americas, Hutchinson, Minn.) were added. The palmitic acid/calcium steartate blend was prepared by grinding together and collecting powders of 300 μm or less from a blend of 20.0005 g palmitic acid and 10.006 g calcium stearate and added to the solid premix.

TABLE 2

| Soil Powder Premix | Grams | % |
|---|---|---|
| Tomato Powder | 20.059 | 18.353 |
| Psyllium Husk | 0.599 | 0.548 |
| Cabbage | 2.145 | 1.963 |
| Spinach Powder | 8.129 | 7.438 |
| Provesta 000 | 40.906 | 37.428 |
| Ohly HCT | 16.628 | 15.214 |
| Palmitic acid/Calcium Stearate (2:1) | 20.827 | 19.056 |

b. Preparation of Liquid Premix

A water premix was made by adding 0.7 mL 1M citric acid, 0.3 mL benzyl alcohol, and 0.125 g sodium benzoate to 70 mL of distilled water. Glycerin (10 g) was weighed in a separate container and added the water premix to bring total weight of water, preservatives and glycerin (i.e., the Liquid premix) to 10 times the weight of the glycerin alone (i.e., 100 g).

c. Preparation of Pasty Artificial Feces

To prepare the artificial feces, water premix, described above, is added to solid premix, described above in Table 1, in a suitable container, to achieve a water content of about 66%. A tongue depressor is used to stir the composition until the composition, which may be a paste, is homogeneous. Cap the container loosely and cover it with a piece of aluminum foil. Place the container in a fully boiling steamer for 40 minutes. Remove the container after the 40 minutes and let cool down to 23°±2.2° C. The test composition is ready for use. If desired, transfer the test composition to a syringe using a sterile tongue depressor for ease of handling. Covered tightly, the Artificial Feces is stable at room temperature for at least 5 days. By stable, it is meant that no appreciable change in ATP counts, hardness, adhesive force, or cohesiveness is expected.

### Examples

The following table shows exemplary soil capture polymers, along with comparative examples and a control of water. The table shows the samples' reduction in percent backscattering at 2 minutes, as determined by the ABM Flocculation and Settling Test Method described herein, the standard deviation of the percent backscattering, the charge density at pH 4, and number and weight average molecular weights. The data show that the inventive soil capture polymers, with the combination of high charge density and high number average molecular weight, are better able to flocculate the ABM and organic soils, as shown by lower % backscattering at 2 minutes. Lower % backscattering at 2 minutes indicates higher flocculation or aggregation of particles, which allows for more effective soil capture by the cleansing implement.

| | | | Charge | | |
|---|---|---|---|---|---|
| | % BS @ | Standard | Density | | |
| Sample | 2 min. | Deviation | meq/g | Mn | Mw |
| Control | 14.3 | 0.1 | | | |
| water | | | | | |
| Comparative | | | | | |
| Examples | | | | | |
| CP903 | 13.5 | 0.1 | 0.4 | | |
| DADMAC, | 18.7 | | Theoretical | 42,000 | 143,000 |
| low MW | | | 5-6 | | |
| DADMAC, | 17.9 | | Theoretical | 85,000 | 361,000 |
| high MW | | | 5-6 | | |
| Hychem | 14.5 | 0.1 | 4.2 | 300,000 | 711,000 |
| Floquat | | | | | |
| TS45SH | | | | | |
| Hychem | 14.4 | 0.1 | Theoretical | 518,000 | 1,492,000 |
| Flobead | | | 5-6 | | |
| DB45VHM | | | | | |
| Inventive | | | | | |
| Soil Capture | | | | | |
| Polymers | | | | | |
| Hychem | 10.5 | 0.3 | 3.4 | 5,313,000 | 5,412,000 |
| CP910 | | | | | |
| Hychem | 8.3 | 0.6 | 3.5 | 5,042,000 | 5,125,000 |
| CP911 | | | | | |
| Hychem | 7.6 | 0.1 | 3.5 | 5,907,000 | 5,997,000 |
| CP911HH | | | | | |
| Hychem | 11.7 | 0.3 | 3.5 | | |
| CP1944 | | | | | |
| Hychem | 8.9 | 0.1 | 2.5 | >1,000,000 | |
| CP9270 | | | | possibly | |
| | | | | 9,000,000 | |
| BASF Zetag | 9.1 | 0.6 | 3.7 | | |
| 8185 | | | | | |
| BASF Zetag | 10.5 | 0.3 | 1.7 | | |
| 8187 | | | | | |
| BASF Zetag | 8.5 | 2.0 | 4.8 | | |
| 8190 | | | | | |
| BASF Zetag | 9.8 | 3.3 | 1.4 | | |
| 8812S | | | | | |
| BASF Zetag | 7.7 | 0.9 | 2.7 | | |
| 8814 | | | | | |
| BASF Zetag | 11.5 | 0.2 | 4.6 | | |
| 8818 | | | | | |
| BASF Zetag | 9.7 | 0.5 | 5 | | |
| 8819 | | | | | |
| BASF Zetag | 10.3 | 0.7 | 4.7 | | |
| 8848FS | | | | | |
| 100% QVI- | 8.1 | 0.1 | 5.9 | 2,404,000 | 3,448,000 |
| Cl | | | | | |
| 100% QVI- | 8.2 | 0.3 | 6.9 | 1,422,000 | 2,068,000 |
| Cl | | | | | |
| 95% QVI- | 9.7 | 1.7 | 6.5 | 1,571,000 | 2,029,000 |
| Cl, 5% VI | | | | | |
| 90% QVI- | 8.3 | 0.5 | 5.8 | 1,617,000 | 2,322,000 |
| Cl, 10% VI | | | | | |
| 90% QVI- | 7.9 | 0.3 | 6.6 | 1,674,000 | 2,385,000 |
| Cl, 10% VI | | | | | |
| 85% QVI- | 8.9 | 0.1 | 6.5 | 1,400,000 | 2,051,000 |
| Cl, 15% VI | | | | | |

The following are examples of lotion compositions. Example 1 is a control lotion composition that does not comprise a soil capture polymer. Examples 2 to 4 are lotion compositions that comprise a soil capture polymer of the present invention.

Lotion Compositions

| Ex. | Ingredients | % w/w |
|---|---|---|
| 1 | Aqua | Q.S. |
| | Disodium EDTA | 0.10 |
| | Sodium Benzoate | 0.18 |
| | Xanthan Gum* | 0.18 |
| | PEG-40 Hydrogenated Castor Oil | 0.44 |
| | Preservative Enhancing Agent^{□} | 0.20 |
| | Citric Acid | 0.53 |
| | Trisodium Citrate | 0.33 |
| | Perfume | 0.07 |
| 2 | Aqua | Q.S. |
| | Disodium EDTA | 0.10 |
| | Sodium Benzoate | 0.24 |
| | Soil Capture Polyme [symbol] | 0.25 |
| | PEG-40 Hydrogenated Castor Oil | 0.44 |
| | Preservative Enhancing Agent^{□} | 0.20 |
| | Citric Acid | 0.53 |
| | Trisodium Citrate | 0.33 |
| | Perfume | 0.07 |
| 3 | Aqua | Q.S. |
| | Disodium EDTA | 0.10 |
| | Sodium Benzoate | 0.18 |
| | Soil Capture Polymer | 0.50 |
| | PEG-40 Hydrogenated Castor Oil | 0.44 |
| | Preservative Enhancing Agent^{□} | 0.20 |
| | Citric Acid | 0.53 |
| | Trisodium Citrate | 0.33 |
| | Perfume | 0.07 |
| 4 | Aqua | Q.S. |
| | Disodium EDTA | 0.10 |
| | Sodium Benzoate | 0.18 |
| | Soil Capture Polymer | 1.00 |
| | PEG-40 Hydrogenated Castor Oil | 0.22 |
| | Preservative Enhancing Agent^{□} | 0.16 |
| | Citric Acid | 0.53 |
| | Trisodium Citrate | 0.33 |

*Xanthan FG as supplied by Jungbunzlauer, Austria.
^{□}Sorbitan caprylate or glyceryl caprylate/caprate as supplied by Clariant under the designation VELSAN ™ SC, by Peter Cremer under the designation CremerCOOR ® GC810, CremerCOOR ® GC8, or IMWITOR ® 742, or by Abitec under the designation CAPMUL ® 708G. [symbol]
ny of the inventive soil capture polymers described in the present invention.

### Soil Adsorption Test (Modified)

The Soil Adsorption Test is adopted herein as disclosed in U.S. Pat. No. 9,212,243, with the following modifications:

In the examples below, a treated substrate of 80/20 polypropylene/viscose was allowed to dry and then cut into 3 inch by 4 inch pieces. It was then added to a vial containing ˜180 mg of ABM and 25 ml of deionized water. The vial was placed on a 180 shaker for 1 minute and then the wipe was removed and wrung out. The amount of soil that does not adhere to the wipe was collected and dried. Subtract this from the starting value to calculate the amount of soil captured by the wipe.

As the data show, the soil capture polymers provide an improvement to the amount of soil captured. In the table below, the first line (control) is for a wipe with a currently marketed lotion that does not contain a soil capture polymer. It captures about 98 mg of soil. The next two lines are comparative examples of wipes comprising non-inventive soil capture polymers in the lotion. These soil capture polymers are either too low in number average molecular weight or charge to provide a backscattering benefit in the turbidity test. Both capture less than 100 mg of soil. The rest of the samples in the table are wipes comprising inventive soil capture polymers in the lotion. These soil capture polymers provide both an improvement (reduction) to the backscattering in turbidity indicating flocculation of ABM and an improvement (increase) in the wipe test indicating capturing more of the soil.

| | Soil Captured (mg) on Wipe |
|---|---|
| Control | 97.7 +/− 1.2 |
| Comparative Hychem flobead | 94.7 +/− 0.2 |
| DB45VHM | |
| Comparative Hychem CP903 | 99.1 +/− 0.9 |
| Hychem CP9270 | 105.1 +/− 4.7 |
| Hychem CP910 | 112.0 +/− 0.7 |
| BASF Zetag 8187 | 110.8 +/− 1.8 |
| Hychem CP911HH | 106.8 +/− 1.2 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

We claim:

1. A lotion composition comprising a soil capture polymer configured to capture organic bodily soils,
the soil capture polymer having a number average molecular weight of at least 1,000,000 and a cationic charge of at least 1 meq/g, and
the lotion pH being from about 3.5 to about 5.5.

2. The lotion composition of claim 1, wherein the soil capture polymer comprises from about 0.15% to about 0.30%, by weight, of the lotion.

3. The lotion composition of claim 1, wherein the soil capture polymer is selected from the group consisting of cationic acrylamide copolymers, quaternary vinylimidazole polymers, vinylimidazole polymers, and combinations thereof.

4. The lotion composition of claim 1, wherein the soil capture polymer is a cationic acrylamide copolymer.

5. The lotion composition of claim 1, wherein the soil capture polymer has a number average molecular weight no greater than 10,000,000.

6. The lotion composition of claim 1, wherein the soil capture polymer has a cationic charge of at most 10 meq/g.

7. The lotion composition of claim 1, wherein the soil capture polymer has a cationic charge of at most 6 meq/g.

8. A wet wipe comprising a lotion composition and a substrate, wherein the lotion composition comprises a soil capture polymer configured to capture organic bodily soils, the soil capture polymer having a number average molecular weight of at least 1,000,000 and a cationic charge of at least 1 meq/g, and the lotion pH being from about 3.5 to about 5.5.

9. The wet wipe of claim 8, wherein the substrate is a nonwoven and comprises fibers.

10. The wet wipe of claim 8, wherein the substrate comprises at least 15% cellulose.

11. The wet wipe of claim 8, wherein the substrate comprises at least 40% cellulose.

12. The lotion composition of claim 1, wherein the soil capture polymer comprises from about 0.20% to about 0.25%, by weight, of the lotion.

13. A lotion composition comprising a soil capture polymer,
the soil capture polymer having a number average molecular weight of at least 1,000,000 and a cationic charge of at least 1 meq/g, and
the soil capture polymer having a percent back-scattering at 2 minutes of at most 12%.

14. The lotion composition of claim 13, wherein the soil capture polymer has a percent back-scattering at 2 minutes of at most 10%.

15. The lotion composition of claim 13, wherein the soil capture polymer comprises from about 0.15% to about 0.30%, by weight, of the lotion.

16. The lotion composition of claim 13, wherein the soil capture polymer is a cationic acrylamide copolymer.

17. The lotion composition of claim 13, wherein the soil capture polymer has a number average molecular weight no greater than 10,000,000.

18. The lotion composition of claim 13, wherein the soil capture polymer has a cationic charge of at most 10 meq/g.

19. A wet wipe comprising a lotion composition and a substrate, the substrate being a nonwoven comprising at least 15% cellulose, wherein the lotion composition comprises a soil capture polymer, the soil capture polymer having a number average molecular weight of at least 1,000,000 and a cationic charge of at least 1 meq/g, and the soil capture polymer having a percent back-scattering at 2 minutes of at most 12%.
